(19) United States
(12) Patent Application Publication
(10) Pub. No.: US 2009/0124702 A1
(43) Pub. Date: May 14, 2009

(54) Pharmaceutical Compositions of Metformin

(76) Inventors: Pechetti Siva Satya Krishna Babu, Hyderabad (IN); Kulkarni Amol, Hyderabad (IN); Gogia Ashish, Hyderabad (IN); Meenakshisunderam Sivakumaran, Hyderabad (IN)

Correspondence Address: Jay R Akhave, 2058 N Mills Ave, # 612, Claremont, CA, 91711, US

(21) Appl. No.: 11/883,031

(22) PCT Filed: Jan. 24, 2006
(86) PCT No.: PCT/IB06/00355

§ 371 Date: Jul. 24, 2007

(30) Foreign Application Priority Data
Jan. 25, 2005 (IN) 57/CHE/2005

(51) Int. Cl. A61K 31/155

(52) U.S. Cl. 514/635

(57) ABSTRACT

The present invention relates to an extended release dosage form of highly water-soluble antidiabetic drug metformin or its pharmaceutically acceptable salts. This invention also relates to methods for preparing the extended release dosage forms of metformin or its pharmaceutically acceptable salts.

### FIELD OF THE INVENTION

The present invention relates to an extended release dosage form of highly water-soluble antidiabetic drug metformin or its pharmaceutically acceptable salts. This invention also relates to methods for preparing the extended release dosage form of metformin or its pharmaceutically acceptable salts.

### BACKGROUND OF THE INVENTION

Metformin hydrochloride is a biguanide derivative used as an oral antidiabetic. Metformin tablets are marketed under the trade name Glucophage and Glucophage XR (extended release).

Metformin hydrochloride has intrinsically poor permeability in the lower portion of the gastrointestinal tract leading to absorption almost exclusively in the upper part of the gastrointestinal tract. Its oral bioavailability is in the range of 40 to 60% decreasing with increasing dosage, which suggests some kind of saturable absorption process, or permeability/transit time limited absorption. It also has a very high water solubility (>300 mg/ml at 25° C.), which leads to difficulty in providing a slow release rate from a formulation and problems in controlling the initial burst of drug from such a formulation. These two difficulties are further compounded by the high unit dose required for metformin hydrochloride.

Typical prior art techniques for creating a controlled release oral dosage form would involve either matrix systems or multi particulate systems. Matrix systems may be formulated by homogeneously mixing drug with hydrophilic polymers, such as hydroxypropylmethylcellulose, hydroxypropylcellulose, polyethylene oxide, carbomer, certain methacrylic acid derived polymers, sodium alginate, or mixtures thereof and compressing the resultant mixture into tablets. Hydrophobic polymers, such as ethyl cellulose, certain polymeric methacrylic acid esters, cellulose acetate butyrate, poly(ethylene-co-vinyl-acetate) may be uniformly incorporated with the above materials to give additional control of release. A further alternative involves embedding drug within a wax-based tablet, by granulation or simply mixing of drug with a wax, such as carnauba wax, microcrystalline wax or commercially available purified fatty acid esters.

Most extended-release forms are designed so that the administration of a single dosage unit provides the immediate release of an amount of drug that promptly produces the desired therapeutic effect and also a gradual and continual release of additional amounts of drug to maintain this level of effect over an extended period of time to overcome frequent or multiple dosing. In this type of dosage form, the design is based on the particular qualities of each individual drug. In the case of Metformin HCl which is a very highly soluble drug, it is imperative to control the release in order to obtain a controlled release formulation.

Other advantages of extended-release products are reduced side effects and increased patient compliance. These advantages relate to the fact that extended-release preparations are designed to maintain the blood concentration of the drug at a desired level over a prolonged period of time thereby reducing the frequency of dosing and thus ensuring patient compliance.

The following patents/publications describes extended/and controlled release compositions of metformin:

U.S. Pat. No. 5,955,106 discloses a composition comprising metformin as the active substance and a hydrocolloid forming retarding agent, wherein the pharmaceutical composition has a residual moisture content of about 0.5-3% by weight.

U.S. Pat. No. 6,340,475 discloses a controlled release gastric retentive oral dosage form comprising a solid polymeric matrix with metformin dispersed therein at a weight ratio of drug to polymer of from about 15:85 to about 80:20. This patent further discloses the polymeric matrix is formed of a polymer selected from the group consisting of poly(ethylene oxide), cellulose, alkyl-substituted celluloses, crosslinked polyacrylic acids, and xanthan gum.

U.S. Pat. No. 6,033,685 discloses a tablet for the controlled release of an active agent comprising (a) a matrix layer comprising an active agent embedded in a non-swelling, non-erodible hydrophobic matrix; (b) a first barrier layer applied to a single face of the matrix layer; and (c) an optional second barrier layer laminated to the opposite face of the matrix layer; wherein the matrix layer comprises up to about 80% active agent and from about 5% to about 80% by weight of nonswellable waxes or polymeric material insoluble in aqueous medium, and the first and second barrier layers independently comprise (1) polymeric material exhibiting a high degree of swelling and gelling in aqueous medium or (2) nonswellable wax or polymeric material insoluble in aqueous medium.

U.S. Pat. Nos. 6,475,521 and 6,660,300 discloses a pharmaceutical formulation comprising (1) an inner solid particulate phase, and (2) an outer solid continuous phase in which particles of the inner solid particulate phase are dispersed and embedded, the particles of the inner solid particulate phase comprising (a) a pharmaceutical having a high water solubility selected from metformin or a pharmaceutically acceptable salt thereof; and (b) an extended release material, and the outer solid continuous phase comprising an extended release material, wherein the total extended release material content in both the inner solid particulate phase and the outer solid continuous phase is within the range from about 25 to about 75% by weight of the pharmaceutical formulation.

U.S. Pat. No. 6,524,618 describes an extended release matrix formulation capable of being directly compressed into tablets comprising (a) about 30 to about 60% of metformin hydrochloride having a particle size of about 150 to about 600 microns, (b) one or more pharmaceutically acceptable polymers selected from the group consisting of polyethylene oxide, hydroxypropyl cellulose, hydroxyethyl cellulose and ethyl cellulose (c) about 5 to about 40% of a pharmaceutically acceptable insoluble filler; (d) about 0.1 to about 3% by weight a glidant; and (e) about 0.1 to about 3% by weight an acceptable lubricant.

US 2003/0187074 describes an oral delivery system for the treatment of non-insulin dependent diabetes mellitus in humans for the controlled release of a biguanide or pharmaceutically acceptable salt thereof, comprising: a pharmaceutically effective amount of a biguanide or pharmaceutically acceptable salt of the biguanide; and a water-insoluble polymeric carrier comprising a water-insoluble polymer; wherein the delivery system provides a pH-independent, controlled release of the biguanide or pharmaceutically acceptable salt thereof over an extended period of time.

U.S. patent Publication 2004/0109891 discloses a sustained-release pharmaceutical composition comprising metformin or a pharmaceutically acceptable salt thereof in an amount of about 100 mg to about 1000 mg; and a sustained-release delivery system comprising xanthan gum in an amount of about 5% to about 60% by weight; locust bean gum in an amount of about 10% to about 70% by weight.

US 2004/0161461 and US 2003/0170302 describes an extended release pharmaceutical tablet comprising: (i) a core comprising by weight, based on the core weight, about, 70% to about 99% metformin and pharmaceutically acceptable excipients; and (ii) a coating surrounding said core, wherein said coating is permeable to metformin, said extended release tablet exhibiting a dissolution profile such that after about 2 hours, from about 7% to about 60% of the metformin is released; after about 4 hours, from about 15% to about 90% of the metformin is released; after about 8 hours, from about 50% to about 100% of the metformin is released; after about 12 hours, more than about 75% of the metformin is released.

WO 03/011255 discloses a gastric retention controlled-drug delivery system comprising (a) a controlled release core comprising a drug, a highly swellable polymer and a gas generating compound, said core being capable of swelling and achieving floatation rapidly while maintaining its physical integrity in gastrointestinal fluids for prolonged periods, and (b) a rapidly releasing coat composition comprising the same drug as in the core and pharmaceutically acceptable excipients, wherein the coating composition surrounds the core such that the system provides a biphasic release of the drug in gastrointestinal fluids. This publication discloses a biphasic extended release dosage form comprising core and coating the core and provides for immediate release and extended release of the drug, which makes the process costlier and complex.

WO 2005/060942 discloses an extended release monophasic dosage form of metformin comprising a matrix of polymer and carbonate.

WO 2005/123134 discloses controlled release composition of metformin comprising hydrophilic polymers and hydrophobic lubricating agents.

The above prior art describes various controlled/extended release compositions of metformin. However, still there is need for developing extended release dosage forms of metformin, which will have release profile comparable to that of marketed dosage forms.

While continuing our efforts to develop extended release formulations of metformin, the inventors of the present invention found that monophasic dosage forms comprising polymer and inorganic silicates resulted in dosage form with dissolution profiles similar to the marketed metformin extended release (Glucophage XR) tablets.

### OBJECTIVE OF THE INVENTION

The object of the present invention is to prepare monophasic extended release pharmaceutical dosage forms of poorly compressible drug, metformin which will have adequate hardness and good reproducibility that releases the drug in a controlled manner over an extended period of time.

Another objective of the present invention is to provide monophasic extended release dosage form, which is uncoated, simple and economic.

Yet another objective of the present invention is to provide extended release solid dosage forms of metformin hydrochloride in such a way that it will comply with the reference product in terms of in vitro parameters like dissolution, disintegration etc and in vivo parameters like bioequivalence.

### SUMMARY OF THE INVENTION

Accordingly, the present invention provides an extended release monophasic dosage form of metformin or its pharmaceutically acceptable salts comprising hydrophilic polymer, an inorganic silicate and one or more pharmaceutically acceptable excipients.

### DETAILED DESCRIPTION OF THE INVENTION

Inorganic silicates are water-swellable clays that have been used for many years in the formulation of tablets, ointments and creams. These are used in oral and topical formulations as suspending and stabilizing agents either alone or in combination with other suspending agents. The advantage of these water-swellable clays is that they produce synergistic rheological effects when mixed with other suspending agents or organic thickeners like xanthan gum, sodium carrageenan, sodium alginate, gum tragacanth, gum arabic, hydroxypropyl guar, sodium carboxymethylcellulose, methyl cellulose etc. The mixtures produce greater viscosity and yield value (therefore greater thickening, and suspending properties and there by improving stability) than those developed by the individual components of the mixture. A water-swellable clay that is particularly effective in combination with xanthan gum is magnesium aluminum silicate and magnesium silicate.

The present invention is based on synergism between organic thickeners with inorganic silicates like magnesium silicate, magnesium tri silicate or magnesium aluminum silicate. The increased viscosity of polymers when combined with silicates provides extended release of metformin from the tablet formulation.

The extended release dosage forms of the present invention are monophasic, which releases the drug for prolonged period of time.

The extended release dosage forms of the present invention comprises drug to polymer plus inorganic silicate in the ratio of 5:0.5 to 1:5.

The extended release dosage forms of the present invention comprises drug and inorganic silicate in the ratio of 1:0.05 to 1:1.

The extended release dosage forms of the present invention comprises drug to polymer in the ratio of 1:0.05 to 1:0.6.

The term pharmaceutically acceptable excipients as used in this invention comprise binders, fillers, lubricants, glidants and the like.

The pharmaceutically acceptable salts of metformin as used here include hydrochloride, hydrobromide and the like.

The inorganic silicates of the present invention are selected from magnesium aluminum silicate, magnesium silicate, magnesium trisilicate, bentonite, calcium silicate, aluminium silicate and the like.

Suitable binders according to the present invention are selected from methylcellulose, hydroxypropyl cellulose, hydroxypropyl methylcellulose, povidone, copovidone, gelatin, gum arabic, ethyl cellulose, polyvinyl alcohol, starch, pregelatinized starch, agar, tragacanth or sodium alginate.

Suitable fillers used according to the present invention are selected from calcium phosphate-dibasic, cellulose-microcrystalline, cellulose powdered, calcium silicate, polyols such as mannitol, sorbitol, xylitol, maltitol, sucrose and combinations thereof.

Suitable lubricants according to the present invention are selected from talc, magnesium stearate, stearic acid, zinc stearate, sodium lauryl sulfate, sodium stearyl fumarate, hydrogenated vegetable oil, glyceryl behenate and suitable glidants include colloidal silicon dioxide or talc.

Suitable hydrophilic polymers according to present invention are selected from polyvinylpyrrolidone, alginate or its salts; xanthan gum, cellulose polymer such as hydroxypropylmethyl cellulose, hydroxypropyl cellulose, hydroxypropylmethyl cellulose phthalate, methylcellulose, carboxymethyl cellulose sodium, hydroxyethyl cellulose and the like; polyethylene oxide, carbopol, pectin, galactomannan, or polyethylene glycol (PEG).

The pharmaceutically acceptable salts of metformin as used here include hydrochloride, hydrobromide and the like.

The present invention provides a simple, non-complex and more economic process for the preparation of an extended release matrix based dosage form, which comprises the steps of:
i) sifting metformin hydrochloride, polymer, and optionally inorganic silicates and fillers,
ii) mixing the sifted materials of step (i) in a rapid mixer granulator,
iii) granulating the blend of step (ii) with a solution of binder,
iv) drying the wet mass of step (iii) in a fluid bed drier,
v) milling the dried granules,
vi) optionally mixing the dried granules with inorganic silicates, polymers and fillers,
vii) lubricating the blend of step (vi) with lubricants and
   compressing the lubricated blend of step (vii) to get extended release tablets of metformin.

The solvent used for preparing binder solution according to the present invention are selected from water, isopropyl alcohol, ethanol, acetone, methylene chloride and the like or mixture thereof.

The following examples further exemplify the inventions and are not intended to limit the scope of the invention. It is obvious to those skilled in the art to find out the composition for other dosage forms and substitute the equivalent excipients as described in this specification or with the one known to the industry.

### EXAMPLE 1

| Ingredients | Quantity |
|---|---|
| Metformin HCl | 750 mg |
| Magnesium Aluminum Silicate | 240 mg |
| Xanthan gum | 60 mg |
| Carbopol 971 P | 96 mg |
| Hydroxypropyl cellulose | 14 mg |
| Magnesium stearate | 10 mg |
| IPA | q.s. |
| Tablet Weight | 1170 mg |

### EXAMPLE 2

| Ingredients | Quantity |
|---|---|
| Metformin HCl | 750 mg |
| Magnesium Aluminum Silicate | 150 mg |
| Xanthan gum | 60 mg |
| Carbopol 971 P | 96 mg |
| Hydroxypropyl cellulose | 14 mg |
| Magnesium stearate | 10 mg |
| IPA | q.s. |
| Tablet Weight | 1080 mg |

The processing steps that are involved in examples 1 and 2 are
i) sifted metformin HCl, magnesium aluminum silicate, xanthan gum, and carbopol 971 P through #40 mesh,
ii) loaded the material of step (i) in RMG and mixed for 15 minutes,
iii) dissolved hydroxypropyl cellulose in sufficient quantity of IPA:water
iv) added the binder solution of step (iii) to dry mix of step (ii) and continued mixing until granules of uniform consistency were obtained,
v) granules were dried, milled and lubricated,
vi) granules of step (v) were compressed to form extended release tablets of metformin.

### EXAMPLE 3

| Ingredients | Quantity |
|---|---|
| Metformin HCl | 750 mg |
| Magnesium Aluminum Silicate | 240 mg |
| Xanthan gum | 60 mg |
| Carbapol 971 P | 96 mg |
| PVP K90 | 14 mg |
| Magnesium stearate | 10 mg |
| IPA | q.s. |
| Tablet Weight | 1170 mg |

### EXAMPLE 4

| Ingredients | Quantity |
|---|---|
| Metformin HCl | 750 mg |
| Magnesium Aluminum Silicate | 150 mg |
| Xanthan gum | 60 mg |
| Carbopol 971 P | 96 mg |
| PVP K90 | 14 mg |
| Magnesium stearate | 10 mg |
| IPA | q.s. |
| Tablet Weight | 1080 mg |

The processing steps that are involved in example 3 and 4 are:
i) sifted metformin HCl, magnesium aluminum silicate, xanthan gum, and carbopol 971 P through #40 mesh,
ii) loaded the material of step (i) in RMG and mixed for 15 minutes,
iii) dissolved polyvinyl pyrrolidone in sufficient quantity of IPA:water
iv) added the binder solution of step (iii) to dry mix of step (ii) and continued mixing until granules of uniform consistency were obtained,
v) unloaded the granules formed and dried,
vi) lubricated the granules of step (v)
vii) compressed the lubricated blend of step (vi) to form extended release tablets of metformin.

### EXAMPLE 5

| Ingredients | Quantity |
|---|---|
| Metformin HCl | 750 mg |
| Magnesium Aluminum Silicate | 240 mg |
| Xanthan gum | 46 mg |
| Carbopol 971 P | 96 mg |
| Hydroxypropyl cellulose | 8 mg |
| Carbopol 71 G | 20 mg |
| Magnesium stearate | 10 mg |
| IPA | q.s. |
| Tablet Weight | 1170 mg |

The processing steps that are involved in example 5 are:
i) sifted metformin HCl, magnesium aluminum silicate, xanthan gum, and carbopol 971P through #40 mesh,
ii) loaded the sifted metformin HCl and Carbopol 971P of step (i) in RMG and mixed for 15 minutes,
iii) dissolved hydroxypropyl cellulose in sufficient quantity of IPA:water
iv) added the binder solution of step (iii) to dry mix of step (ii) and continued mixing until granules of uniform consistency were obtained,
v) unloaded the granules formed and dried,
vi) sifted the granules of step (v) through # 20 sieve,
vii) added the sifted magnesium aluminium silicate, xanthan gum of step (i) and carbopol 71 G to the sifted granules of step (vi),
viii) compressed the lubricated blend of step (vi) to form extended release tablets of metformin.

### EXAMPLE 6

| | Quantity | |
|---|---|---|
| Ingredient | | |
| Metformin HCl | 750 | mg |
| Carbopol 971P | 96 | mg |
| Hdroxypropyl cellulose | 8 | mg |
| IPA | q.s | |
| Extragranular | | |
| Carbopol 71 G | 20 | mg |
| Xanthan gum | 46 | mg |
| Magnesium Trisilicate | 297.7 | mg |
| Magnesium Stearate | 12.30 | mg |
| Total | 1230 | mg |

The processing steps that are involved in example 6 are:
i) sifted metformin HCl, and carbopol 971P through mesh # 40 and mixed for 10 minutes,
ii) dissolved hydroxypropyl cellulose in isopropyl alcohol,
iii) granulated the contents of step (i) with binder solution of step (ii),
iv) dried the granules of step (iii),
v) sifted magnesium trisilicate, carbopol 71G, and xanthan gum through mesh # 30 and mixed with the dried granules of step (iv),
vi) lubricated the contents of step (v) and
vii) compressed the blend of step (vi) into tablets.

### EXAMPLE 7

| | Quantity | |
|---|---|---|
| Ingredient | | |
| Metformin HCl | 750 | mg |
| Carbopol 971P | 116 | mg |
| Hdroxypropyl cellulose | 8 | mg |
| IPA | q.s | |
| Extragranular | | |
| Xanthan gum | 46 | mg |
| Magnesium Trisilicate | 297.7 | mg |
| Magnesium Stearate | 12.30 | mg |
| Total | 1230 | mg |

The processing steps that are involved in example 7 are:
i) sifted metformin HCl, and carbopol 971P through mesh # 40 and mixed for 10 minutes,
ii) dissolved hydroxypropyl cellulose in isopropyl alcohol,
iii) granulated the contents of step (i) with binder solution of step (ii),
iv) dried the granules of step (iii),
v) sifted magnesium trsilicate, and xanthan gum through mesh # 30 and mixed with the dried granules of step (iv),
vi) lubricated the contents of step (v) and
vii) compressed the blend of step (vi) into tablets.

### EXAMPLE 8

| | Quantity | |
|---|---|---|
| Ingredient | | |
| Metformin HCl | 750 | mg |
| Magnesium Trisilicate | 140.85 | mg |
| Carbopol 971P | 116 | mg |
| Hdroxypropyl cellulose | 16 | mg |
| IPA | q.s | |
| Extragranular | | |
| Xanthan gum | 46 | mg |
| Magnesium Trisilicate | 148.85 | mg |
| Magnesium Stearate | 12.30 | mg |
| Total | 1230 | mg |

The processing steps that are involved in example 8 are:
i) sifted metformin HCl, magnesium trisilicate, and carbopol 971P through mesh # 40 and mixed for 10 minutes,
ii) dissolved hydroxypropyl cellulose in isopropyl alcohol,
iii) granulated the contents of step (i) with binder solution of step (ii),
iv) dried the granules of step (iii),
v) sifted magnesium trsilicate, and xanthan gum through mesh # 30 and passed through roler compacter and collected the flakes,
vi) flakes of step (v) were passed mesh # 18 and mixed with the dried granules of step (iv),
vii) lubricated the blend of step (vi) and compressed into tablets.

### Comparative Dissolution Profile of Metformin HCl Extended Release Tablets with Glucophage XR (BMS)

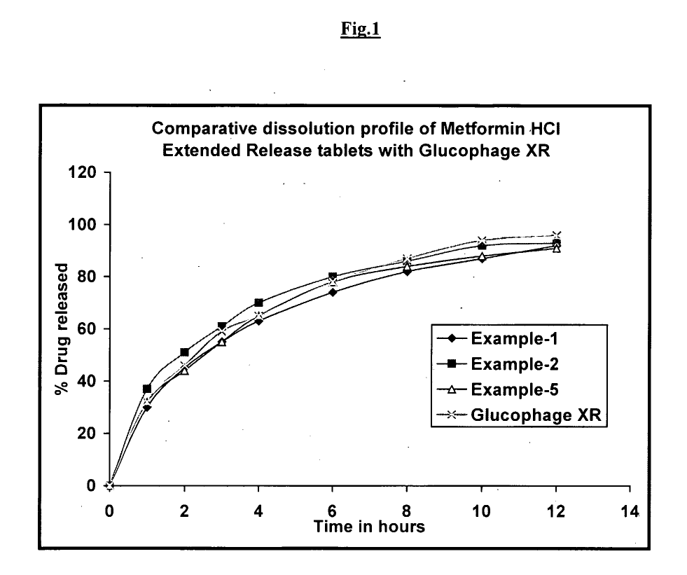

Table 1 given below shows the dissolution profile of extended release tablets of metformin carried out in pH 6.8 Phosphate buffer as medium using—USP-I (Basket) Apparatus, @100 rpm speed. The results were represented graphically in FIG. 1

TABLE 1

| | % Drug released | | | |
|---|---|---|---|---|
| Time in | | | | Glucophage XR |
| hours | Example 1 | Example 2 | Example 5 | (750 mg) |
| 1 | 30 | 37 | 32 | 32 |
| 2 | 45 | 51 | 44 | 46 |
| 3 | 55 | 61 | 55 | 59 |
| 4 | 63 | 70 | 65 | 65 |
| 6 | 74 | 80 | 78 | 78 |
| 8 | 82 | 86 | 84 | 87 |
| 10 | 87 | 92 | 88 | 94 |
| 12 | 92 | 93 | 91 | 96 |

### Bio Equivalence Study

To compare the rate and extent of absorption of Metformin HCl 750 mg (TEST) extended release dosage form of the present invention with Glucophage XR 750 mg (REFERENCE) of Bristol-myers Squibb, USA (Reference), a randomized, cross-over, single-dose oral comparative bioavailability study was conducted in 12 healthy, adult, male, human subjects under fed conditions. The results of this study are as given in Table 2:

TABLE 2

| | Ratio | | | |
|---|---|---|---|---|
| Dependant | [% Ref] | CI_90_Lower | CI_90_Upper | Power |
| Ln(AUCINF_obs) | 93.71 | 85.28 | 102.97 | 0.99 |
| Ln (AUClast) | 94.02 | 83.66 | 105.66 | 0.93 |
| Ln (Cmax) | 107.66 | 96.62 | 119.97 | 0.96 |

The study results indicate that the pharmacokinetic parameters for Test and Reference are falling with in the 80-125% FDA acceptance range. Based on these results, the Metformin HCl extended release dosage form of the present invention and Glucophage XR tablets are meeting bioequivalent criteria under fed conditions.

1. An extended release monophasic dosage form of metformin or its pharmaceutically acceptable salts comprising hydrophilic polymer, an inorganic silicate and one or more pharmaceutically acceptable excipients.

2. The extended release dosage form as claimed in claim 1, wherein drug to polymer plus inorganic silicate is present in the ratio of 5:0.5 to 1:5.

3. The extended release dosage form as claimed in claim 1, wherein drug and inorganic silicate is present in the ratio of 1:0.05 to 1:1.

4. The extended release dosage form as claimed in claim 1, wherein drug to polymer is in the ration of 1:0.05 to 1:0.6.

5. The extended release dosage form as claimed in claim 1, wherein the excipients comprise binders, fillers, lubricants or glidants.

6. The extended release dosage form as claimed in claim 1, wherein the hydrophilic polymer is selected from polyvinylpyrrolidone; alginate or its salts; xanthan gum, cellulose polymer such as hydroxypropylmethyl cellulose, hydroxypropyl cellulose, hydroxypropylmethyl cellulose phthalate, methylcellulose, carboxymethyl cellulose sodium or hydroxyethyl cellulose; polyethylene oxide, carbopol, pectin, cyclodextrins, galactomannan, polyethylene glycol or combination thereof.

7. The extended release dosage form as claimed in claim 1, wherein the inorganic silicate is selected from magnesium aluminum silicate, magnesium silicate, magnesium trisilicate, bentonite, calcium silicate or aluminium silicate.

8. The extended release dosage form as claimed in claim 1, wherein binder is selected from methylcellulose, hydroxypropyl cellulose, hydroxypropyl methylcellulose, povidone, copovidone, gelatin, gum arabic, ethyl cellulose, polyvinyl alcohol, starch, pregelatinized starch, agar, tragacanth or sodium alginate.

9. The extended release dosage form as claimed in claim 1, wherein the filler is selected from calcium phosphate-dibasic, cellulose-microcrystalline, cellulose powdered, calcium silicate, polyols such as mannitol, sorbitol, xylitol, maltitol, sucrose and combinations thereof.

10. The extended release dosage form as claimed in claim 1, lubricant is selected from talc, magnesium stearate, stearic acid, zinc stearate, sodium lauryl sulfate, sodium stearyl fumarate, hydrogenated vegetable oil, glyceryl behenate.

11. A process for the preparation of an extended release dosage form of metformin or its pharmaceutically acceptable salts comprising polymer and an inorganic silicate and one or more pharmaceutically acceptable excipients, which comprises the steps of:
i) sifting metformin hydrochloride, polymer, and optionally inorganic silicates and fillers,
ii) mixing the sifted materials of step (i) in a rapid mixer granulator,
iii) granulating the blend of step (ii) with a solution of binder,
iv) drying the wet mass of step (iii) in a fluid bed drier,
v) milling the dried granules,
vi) optionally mixing the dried granules with inorganic silicates, polymers and fillers,
vii) lubricating the blend of step (vi) with lubricants and
vi) compressing the lubricated blend of step (vii) to get extended release tablets of metformin.
